(19) United States
(12) Patent Application Publication
(10) Pub. No.: US 2021/0186887 A1
(43) Pub. Date: Jun. 24, 2021

(54) MOISTURE RESISTANT PROBIOTIC GRANULE AND METHODS OF PRODUCING THE SAME

(71) Applicant: POLYCAPS HOLDINGS LTD., KFAR SABA (IL)

(72) Inventors: ADEL PENHASI, MODIIN (IL); ISRAEL BALUASHVILI, JERUSALEM (IL)

(21) Appl. No.: 17/048,614

(22) PCT Filed: Apr. 20, 2019
(86) PCT No.: PCT/IL2019/050454

§ 371 Date: Oct. 19, 2020

Related U.S. Application Data
(60) Provisional application No. 62/660,388, filed on Apr. 20, 2018.

(51) Int. Cl. A61K 9/50; A61K 35/745

(52) U.S. Cl. CPC A61K 9/5063; A61K 35/745; A61K 9/5089; A61K 9/5042; A61K 9/5021

(57) ABSTRACT

There is provided a probiotic microcapsule comprising a core comprising probiotic microorganisms; and a coating layer comprising a hybrid solid dispersion comprising an edible fatty molecule evenly dispersed within a water-soluble film forming polymer and an edible mediator, wherein said edible mediator is starch octenyl succinate.

### FIELD OF THE INVENTION

The present invention relates to the fields coating of probiotic granules, and more particularly, to probiotic granules intended to be mixed in foodstuff with a relatively high level of water activity.

### BACKGROUND OF THE INVENTION

Protection of nutritional and nutraceutical dosage forms from environmental moisture is important when the active material is adversely affected by the presence of water. The negative effects of moisture may occur during common production processes such as processes that involve wet granulation and/or coating. Alternatively, the moisture may damage the active material during storage and negatively affect the shelf life of a final product.

With regard to probiotics, moisture is especially a crucial factor in stability and shelf life of many probiotic bacteria. In many cases the exposure of such probiotics to a certain level of humidity may result in deactivation of bacteria. As a result, the range of food products, especially those with a high level of water activity, in which such bacteria can be incorporated will be limited and the shelf life will be considerably shortened.

Common approaches aimed to limit the damage to the active material, include packaging of the dosage forms containing the moisture sensitive active material in different packaging elements, such as microcapsules, tablets, capsules and the like. However, especially in places where climate is very humid, the special packaging does not provide a complete moisture protection because of the moisture captured inside the above mentioned packaging. Another way to prevent or diminish the damage that may be caused by moisture and to reduce the need for special packaging is to coat the solid dosage forms with materials which have moisture barrier properties.

Such materials have essentially a low water vapour permeation (WVP) or a low water vapor transition rate (WVTR). These coatings usually do not affect the basic properties of the dosage forms such as the disintegration time and the release profile of the active material. Examples of moisture sensitive drugs include atorvastatin, ranitidine, temazepam, most vitamins, numerous herbals, unsaturated fatty acids and probiotic bacteria. The damage that may occur due to moisture may include, for example, degradation of active material by hydrolysis, destruction of probiotic bacteria or significant reduction in CFU (colony forming unites) value, changes in the appearance of the dosage form on storage, changes in the disintegration and/or dissolution times of the dosage form. Moisture barrier coatings are thus applied to protect the dosage form from such damages.

In order to achieve a moisture barrier coating, usually a hydrophobic water insoluble polymer is used. The polymers generally employed for this purpose are polyvinyl acetate, zein, shellac, cellulose acetate phthalate (CAP), EUDRAGIT® E 100 which is a cationic copolymer based on dimethylaminoethyl methacrylate, butyl methacrylate, and methyl methacrylate with a ratio of 2:1:1, ethylcellulose (EC) and the like. Such polymers, however, prolong the disintegration of the dosage form in the body after administration and thus delay the release of active materials or probiotic bacteria. Likewise, coating with these polymers necessitates the involvement of use of organic solvent which is not desired because such a process enforces additional expenses relating to air conditioning equipment, anti-explosion provisions, and the like to safely handle such materials. Another way to achieve a moisture barrier coating is a combination of a water soluble polymer with lipophilic substances. The hydrophobic or lipophilic substances particles will be embedded in water soluble film after coating or film formation. Although the presence of lipophilic substance particles in the film may reduce the water vapour transition, they cannot cover all the area of the film, and thus water vapour can still easily penetrate through the spaces between the particles.

Probiotics in general, are microorganisms susceptible to moisture, and there are specific strains which are more vulnerable than others.

BB-12 is a specific strain of Bifidobacterium lactis which is cultivated by Chris Hansen and LGG (a specific strain of Lactobacillus rhamnosus (LGG) which is cultivated by Chris Hansen), are considered highly susceptible to moisture and especially to an environment with a high-water activity (aw). As a result, the survivability of these bacteria decreases considerably in a very short time when they are combined in foodstuff with a relatively high level of water activity.

Bifidobacteria are anaerobic, rod shaped gram-positive bacteria that normally inhabit the human's, infant's and adult's, colon (Simon G L and Gorbach S L. Intestinal flora in health and disease. Gastroenterol, 1984; 86: 174-193). Beneficial effects of bifidobacteria, including improvement of intestinal flora by preventing colonization of pathogens, activation of the immune system, increased protein digestion and amelioration of diarrhea or constipation have been reported (Ishibashi N and Shimamura S. Bifidobacteria: Research and development in Japan. Food Technol, 1993; 6: 126-136). Bifidobacteria have been consumed in fermented foods for decades and current commercial strains include Bifidobacterium animalis (B. animalis) ssp. lactis strain Bf-6, Bifidobacterium lactis (B. lactis) BB-12, B. lactis DR10 (HNO19), Bifidobacterium longum (B. longum) BB536, B. breve Yakult, B. breve SBT-2928, and B. breve C50. In the United States, various Bifidobacterium species have been determined to be GRAS for use in conventional foods and infant formulas, including: B. animalis ssp. lactis Bf-6 for use in selected foods (GRN 377; 1011 cfu/serving of conventional foods); B. lactis Bb-12 for use in infant formulas for four months-of-age and older (GRN 49; 107-108 cfu/g infant formula) and B. longum BB536 for use in selected foods and infant formulas (GRN 268; 1010 cfu/serving of conventional foods; 1010 cfu/g of term infant formula). The minimum level of viable Bifidobacteria in commercial dairy products to exert beneficial effects on human health is known to be approximately 105˜107 CFU/ ml (Cui J H, Shim J M, Lee J S, et al. Gastric acid resistance of Lactobacilli and Bifidobacteria in commercial drink and lipid yogurts. Kor J Microbiol, 2000; 36: 161-165). Recently, there has been an increasing motivation in the application of Bifidobacteria in infant foods, especially powdered infant formula (PIF) (Duncker, 2013). This is mainly for maintaining beneficial micro-flora in infant especially upon antibiotics' treatment which damages the natural micro-flora (Morinaga Milk Industry Co. Ltd. Bifidobacteria & Health.Japan: August 1997, pp. 4).

Bifidobacterium breve M-16V (B. breve) is a Y-shaped, Gram-positive anaerobic bacterium. This organism was deposited with the Belgian Co-ordinated Collections of Microorganisms (BCCM) and designated LMG 23729. B. breve has been detected in the stools of infants and adults. B. breve M-16V was first commercially available in Japan in 1976. The original frozen culture of B. breve M-16V is tightly controlled to ensure purity and stability of the strain. Product specifications assure that B. breve M-16V is suitable for use in food, including term infant formula, exempt term infant formula, and medical foods. Finished products made with B. breve M-16V cultures reproducibly meet compositional standards and comply with limits on contaminants appropriate for food-grade ingredients. B. breve M-16V meets the safety standards enumerated by the Food and Agriculture Organization of the United Nations/World Health Organization's (FAO/WHO) guidelines for the evaluation of microbes for probiotic use in foods. Results show that B. breve M-16V is not toxic or pathogenic and is therefore safe for use in foods. Bifidobacterium animalis subsp. Lactis (also commercially known as BB-12) will be referred to herein as “Bifidobacterium” is a catalase-negative, rod-shaped bacterium. It was deposited in the cell culture bank of Chr. Hansen in 1983. At the time of isolation, Bifidobacterium was considered to belong to the species Bifidobacterium bifidum. Modern molecular classification techniques reclassified BB-12 as Bifidobacterium animalis and later to a new species Bifidobacterium lactis. The species B. lactis was later shown not to fulfill the criteria for a species and was instead included in Bifidobacterium animalis as a subspecies. Today, BB-12 is therefore classified as Bifidobacterium animalis subsp. lactis. Despite a change in the name over the years, the strain BB-12 has not changed.

Bifidobacterium originates from Chr. Hansen's collection of dairy cultures. It is a strain that was specially selected by Chr. Hansen for the production of probiotic dairy products. Bifidobacterium has been used in infant formula, dietary supplements and fermented milk products worldwide. This strain is technologically well suited, expressing fermentation activity, high aerotolerance, good stability and a high acid and bile tolerance, also as freeze-dried products in dietary supplements. Furthermore, Bifidobacterium does not have adverse effects on taste, appearance or on the mouth feel of the food and is able to survive in the probiotic food until consumption.

Chr. Hansen is exclusively the producer of Bifidobacterium, which is one of the most important and most widely studied probiotic bacteria strain for all time. This specific strain is currently used in different dietary supplements and food products, such as infant formula and fermented milk products.

Lactobacillus rhamnosus is a short Gram-positive heterofermentative facultative anaerobic non-spore-forming rod that often appears in chains. While Lactobacillus rhamnosus GG (ATCC 53103) is able to survive the acid and bile of the stomach and intestine, is claimed to colonize the digestive tract, and to balance intestinal microflora, evidence suggests that Lactobacillus rhamnosus is likely a transient inhabitant, and not autochthonous. Regardless, it is considered a probiotic useful for treatment of various maladies, as it works on many levels. Lactobacillus rhamnosus GG binds to the gut mucosa.

### BRIEF DESCRIPTION OF THE DRAWINGS

The present invention will become fully understood from the detailed description given herein below and the accompanying drawings, which are given by way of illustration and example only and not intended to limit the scope of the invention.

FIG. 1 depicts a graph demonstrating the water content of microencapsulated bacteria compared to uncoated bacteria, kept at the same conditions where the on-going stability test is being performed, was also measured over time using a Loss-On-Dry (LOD) method.

### SUMMARY OF THE INVENTION

According to some demonstrative embodiments, there is provided herein a probiotic microcapsule comprising: a core comprising probiotic microorganisms; and a coating layer comprising a hybrid solid dispersion comprising an edible fatty molecule evenly dispersed within a water-soluble film forming polymer and an edible mediator, wherein said edible mediator is starch octenyl succinate.

According to some demonstrative embodiments, the water-soluble film forming polymer may be hydroxypropyl starch.

According to some embodiments, the probiotic microorganisms may include Bifidobacterium.

According to some embodiments, the edible fatty molecule may be selected from the group including lauric acid, myristic acid, palmitic acid, palmitate, palmitoleate, hydroxypalmitate, arachidic acid, oleic acid, stearic acid, sodium stearat, calcium stearate, magnesium stearate, hydroxyoctacosanyl hydroxystearate, oleate esters of long-chain, esters of fatty acids, fatty alcohols, esterified fatty diols, hydroxylated fatty acid, hydrogenated fatty acid (saturated or partially saturated fatty acids), partially hydrogenated soybean, partially hydrogenated cottonseed oil, aliphatic alcohols, phospholipids, lecithin, phosphathydil cholin, triesters of fatty acids, coconut oil, hydrogenated coconut oil, cacao butter; palm oil; fatty acid eutectics; mono and diglycerides, poloxamers, block-co-polymers of polyethylene glycol and polyesters or a combination thereof.

According to some preferred embodiments, the edible fatty molecule may be cocoa butter and/or stearic acid.

According to some embodiments, the hybrid solid dispersion may be a single hybrid solid dispersion.

### DETAILED DESCRIPTION OF THE INVENTION

According to some demonstrative embodiments, there is provided a microcapsule (also referred to herein as a granule) comprising a core comprising probiotics and at least one coating layer protecting the probiotics from moisture.

According to some demonstrative embodiments, the granule may be incorporated into food stuff, mainly liquid foods including for example, water based foods, liquid infant formulas, yogurt, dairy products, nectars, fruit juices and the like.

According to some demonstrative embodiments, the microcapsule may preferably be incorporated into foodstuff with a relatively high level of water activity.

According to some demonstrative embodiments, the probiotics may include any suitable live microorganisms intended to provide health benefits when consumed, generally by improving or restoring the gut flora, for example, a genus selected from Lactobacillus, Bifidobacterium, Bacillus.

According to some embodiments, the probiotics are preferably Bifidobacterium and/or LGG bacteria.

According to some demonstrative embodiments, the at least one coating may include a specific sealing film coating comprising a hybrid solid dispersion in which an edible fatty molecule is evenly dispersed within a water-soluble film forming polymer using, for example, an edible mediator.

According to some demonstrative embodiments, the fatty molecule may include any suitable edible fatty acid, including, for example, lauric acid, myristic acid, palmitic acid, palmitate, palmitoleate, hydroxypalmitate, arachidic acid, oleic acid, stearic acid, sodium stearat, calcium stearate, magnesium stearate, hydroxyoctacosanyl hydroxystearate, oleate esters of long-chain, esters of fatty acids, fatty alcohols, esterified fatty diols, hydroxylated fatty acid, hydrogenated fatty acid (saturated or partially saturated fatty acids), partially hydrogenated soybean, partially hydrogenated cottonseed oil, aliphatic alcohols, phospholipids, lecithin, phosphathydil cholin, triesters of fatty acids, coconut oil, hydrogenated coconut oil, cocoa butter; palm oil; fatty acid eutectics; mono and diglycerides, poloxamers, block-co-polymers of polyethylene glycol and polyesters or a combination thereof.

According to some embodiments, the fatty molecule is preferably cocoa butter and/or stearic acid.

According to some demonstrative embodiments, the water-soluble film forming polymer may include, for example, one or more of Hydroxypropyl starch (HPS), poly-N-substituted acrylamide derivative, polypropyleneoxide, polyvinylmethylether, partially-acetylated product of polyvinyl alcohol, Methylcellulose (MC), hydroxylpropylcellulose (HPC), methylhydroxyethylcelluloce (MHEC), hydroxylpropylmethylcellulose (HPMC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), hydrophobically modified hydroxyethylcellulose (NEXTON), amylose, amylopectin, Poly(organophosphazenes), xyloglucan, synthetic elastin derivative proteins and any of the above polymers further substituted with a hydrophilic or hydrophobic monomer.

According to some embodiments, the poly-N-substituted acrylamide derivative may include one or more of poly(N-isopropylacrylamide) (PNIPAM), Poly-N-acryloylpiperidine, poly(N,N-diethylacrylamide) (PDEAAm), poly(N-vinlycaprolactam) (PVCL), poly[2-(dimethylamino)ethyl methacrylate] (PDMAEMA), poly(ethylene glycol) (PEG), poly(ethylene oxide) (PEO), PEG methacrylate polymers (PEGMA), Poly-N-propylmethacrylamide, Poly-N-isopropylacrylamide Poly-N-diethylacrylamide, Poly-N-isopropylmethacrylamide, Poly-N-cyclopropylacrylamide, Poly-N-acryloylpyrrolidine, Poly-N,N-ethylmethylacrylamide, Poly-N cyclopropylmethacrylamide, Poly-N-ethylacrylamide, poly-N-substituted methacrylamide derivatives, copolymers comprising an N-substituted acrylamide derivative and an N-substituted methacrylamide derivative, and a copolymer of N-isopropylacrylamide and acrylic acid.

In some demonstrative embodiments, the hydrophilic monomer may include one or more of N-vinyl pyrrolidone, vinylpyridine, acrylamide, methacrylamide, N-methylacrylamide, hydroxyethylmethacrylate, hydroxymethylacrylate, hydroxyethylacrylate, hydroxymethyl-methacrylate, methacrylic acid and acrylic acid having an acidic group, and salts of these acids, vinylsulfonic acid, styrenesulfonic acid, derivatives having a basic group such as N,N-dimethylaminoethylmethacrylate, N,N-diethylaminoethyl methacrylate, N,N-dimethylaminopropyl-acrylamide, and salts of these derivatives.

In some demonstrative embodiments, the hydrophobic monomer may include one or more of ethylacrylate, methylmethacrylate, and glycidylmethacrylate; N-substituted alkymethacrylamide derivatives such as N-n-butylmethacrylamide; vinylchloride, acrylonitrile, styrene, vinyl acetate.

According to some embodiments, the water soluble film forming polymer is HPS and/or HPC.

According to some embodiments, the edible mediator may include, for example, Starch Octenyl Succinate (SOS).

According to some demonstrative embodiments, the edible mediator may reduce the interfacial tension between the water-soluble film forming polymer and the edible fatty molecule. According to some embodiments, reducing the surface tension may create a tight integration between the water-soluble film forming polymer and the edible fatty molecule, for example, assuring the formation of a uniform, firm and integer film coat surrounding the core of the microcapsules.

According to some embodiments, the Starch Octenyl Succinate comprises at least two portions which enable the surprising adherence of a hydrophilic component and a hydrophobic component. Specifically, Starch Octenyl Succinate in some embodiments may include a hydrophilic portion which may adhere to the water soluble film forming polymer, and at least one hydrophobic portion which adheres to the edible fatty molecule.

According to some embodiments, these unique features of Starch Octenyl Succinate allow for the creation of a single hybrid solid dispersion which acts as a moisture barrier, preventing the penetration of humidity into the core of said microcapsule.

According to some embodiments, the hybrid solid dispersion provides an essentially uniform coating which repels water from penetrating into the core.

According to some embodiments, a single hybrid solid dispersion may provide superior properties, for example, in comparison to separate protective layer, e.g., separate layers may have a higher potential for penetration of humidity for example, during layering and/or due to a non-homogeneous coverage of each layer.

According to some demonstrative embodiments, the probiotics may be present in an amount ranging between 10-60% w/w from the weight of the microcapsule.

According to some embodiments of the present invention, the core may further comprise a filler. Examples of fillers include, but are not limited to, microcrystalline cellulose, a sugar, such as lactose, glucose, galactose, fructose, or sucrose; dicalcium phosphate; sugar alcohols such as sorbitol, manitol, mantitol, lactitol, xylitol, isomalt, erythritol, and hydrogenated starch hydrolysates; corn starch; potato starch; sodium carboxymethycellulose, ethylcellulose and cellulose acetate, or a mixture thereof. In a preferred embodiment, the filler is lactose.

According to some demonstrative embodiments, the filler may be present in an amount ranging between 60-70% w/w from the weight of the microcapsule.

According to some embodiments of the present invention, the core may further comprise a binder.

Examples of binders include, by way of non-limiting example, Povidone (PVP: polyvinyl pyrrolidone), Copovidone (copolymer of vinyl pyrrolidone and vinyl acetate), polyvinyl alcohol, low molecular weight hydroxypropylmethyl cellulose (HPMC), low molecular weight hydroxypropyl cellulose (HPC), low molecular weight hydroxymethyl cellulose (MC), low molecular weight sodium carboxy methyl cellulose, low molecular weight hydroxyethylcellulose, low molecular weight hydroxymethylcellulose, cellulose acetate, gelatin, hydrolyzed gelatin, polyethylene oxide, acacia, dextrin, maltodextrin, starch, and water soluble polyacrylates and polymethacrylates, low molecular weight ethylcellulose or a mixture thereof. In a preferred embodiment, the binder is maltodextrin.

According to some demonstrative embodiments, the binder may be present in an amount ranging between 10-15% w/w from the weight of the microcapsule.

According to some demonstrative embodiments, the microcapsule may be incorporated into foodstuff possessing high water activity, for example, without harming the probiotics contained within the microcapsule.

Water activity or a_{w} is the partial vapor pressure of water in a substance divided by the standard state partial vapor pressure of water. In the field of food science, the standard state is most often defined as the partial vapor pressure of pure water at the same temperature. Pure distilled water has a water activity of exactly one.

According to some demonstrative embodiments, there is provided herein a microencapsulation formulation which provides susceptible probiotic bacteria with superior protection against humidity to hinder their deactivation and thus lengthening the shelf life. This in turn can extend the shelf life of the products in which the bacteria will be incorporated even at high temperatures.

This preliminary study provides a robust proof of concept for this specific microencapsulation designed to protect the sensitive probiotics bacteria in an environment with relatively high humidity at room temperature. The feasibility of the technology was proven by exposing the microencapsulated probiotic bacteria to air at room temperature for a prolonged time and the water content using an LOD method. Likewise, the microencapsulation process was found to be a totally safe where the initial number of the bacteria was kept similar to that of the initial one.

According to some demonstrative embodiments, there is provided herein a method for preparing a microcapsule for the protection of probiotics from moisture, wherein the method includes:

Preparing a microcapsule core comprising Bifidobacterium;

Coating the core with at least one coating comprising low water vapor transmission rate (WVTR) values; wherein the at least one coating comprises Hydroxypropyl starch (HPS) Cocoa Butter (CB) and Starch Octenyl Succinat.

According to some embodiments, the method may include preparing a 5% w/w solution of HPS by adding HPS to heated distilled water (85-90° C.);

adding Starch Octenyl Succinat to heated distilled water and stiffing continually until a homogeneous clear solution is obtained; and pre-melting Cocoa Butter while stirring using a mechanical stirrer.

According to some embodiments, the cocoa butter was pre-melted at 50° C.

According to some embodiments, the resulting Starch Octenyl Succinat solution and the melted CB are homogenized into the 5% w/w of HPS solution using a homogenizer

### C) Methods

### i) Films Preparation: Based on Hydroxypropyl Starch (HPS) as the Film Forming Polymer

Different film formulations were prepared and characterized by water vapor transmission rate (WVTR). HPS, Cocoa Butter (CB) and Starch Octenyl Succinat (also commercially referred to as Emfix) were accurately weighed to obtain a certain ratio of HPS: CB: Starch Octenyl Succinat. Table 2 presents different film's formulations, possessing various ratios of HPS: CB: Starch Octenyl Succinat, prepared in the present study. First a desired amount of HPS was added to heated distilled water (85-90° C.), while mixing using a magnetic stirrer, to make a 5% w/w of the polymer. The stiffing continued until a homogeneous clear solution was obtained (15-30 min at 85-90° C.). Then a desired amount of Starch Octenyl Succinat was added into a heated distilled water, the stiffing continued until a homogeneous clear solution was obtained. A Cocoa Butter was pre-melted at 50° C., while stiffing using a mechanical stirrer. The resulting Starch Octenyl Succinat solution and the melted CB were then homogenized into a 5% w/w of HPS solution using a homogenizer for 90 sec, the homogenizer speed was 3500 RPM (Homogenizer, HSIANG TAI MACHINERY INDUSTRY, model: HG-300 maximum speed—26000 rpm). The latter dispersion was then cooled down to 32-36° C. and kept stirred for additional 20 minutes to allow complete dissolution of the polymer.

### EXAMPLES

### Example 1—Core and Coating Composition Embodiments—(Solution-Based Core Formation)

Examples of both the core and coating are listed below in Table 1.

TABLE 1
core and coating examples

| Material Name | Product Name | Manufacture | Use for | % w/w |
|---|---|---|---|---|
| Bifidobacterium | Probio-Tec- | Ch. Hans. | Bacteria | 10-20 |
| | Bifidobacterium | | | |
| | Blend-100 V1 | | | |
| Lactose | Pharmatose 100M | DFE Pharma | Filler | 61-67 |
| | Lactose Monohydrate, | | | |
| | sieved | | | |
| Maltodextrin | MALDEX G190 | TEROSE | Binder | 12-13 |
| | PHARMA | SYRAL | | |
| Hydroxypropyl | | — | Coating | — |
| starch (HPS) | | | | |
| Cocoa Butter | Cocoa Butter | Lubeca | Coating | — |
| (CB) | | Germany | | |
| Starch Octenyl | | — | Coating | — |
| Succinate | | | | |
| (SOS) | | | | |

### Example 2—Method of Microencapsulation of Core Comprising Bifidobacterium

In one embodiment, a method of microencapsulation is as follows.

### A) Formulation of the Film Coat

In order to find the right formulation providing the highest barrier capability, various combinations of the coating components were prepared and tested for water vapor transmission rate (WVTR) in Versaperm Ltd. Laboratories (Maidenhead, UK).

### B) Preparation of Bifidobacterium Microencapsulation

First the core, including Bifidobacterium, was prepared using a formulation as desired. The resulting core was then coated using those specific coating formulations which presented the lowest WVTR values. Likewise, the microencapsulation process was planned to find out whether a specific coating formulation is also able to coat the core during the process in spite of its ability to form a free film which was needed for WVTR test performance. The results from this part of experiment showed that the coating process can take place very smooth and fast, where still the particle size and particle size distribution can perfectly be in the required range. The LOD (Loss of drying) values can be controlled and be adjusted in a range enabling the long-lasting stability of the bacteria.

### C) Methods

### i) Films Preparation: Based on Hydroxypropyl Starch (HPS) as the Film Forming Polymer

Different film formulations were prepared and characterized by water vapor transmission rate (WVTR). HPS, Cocoa Butter (CB) and Starch Octenyl Succinat (also commercially referred to as Emfix) were accurately weighed to obtain a certain ratio of HPS: CB: Starch Octenyl Succinat. Table 2 presents different film's formulations, possessing various ratios of HPS: CB: Starch Octenyl Succinat, prepared in the present study. First a desired amount of HPS was added to heated distilled water (85-90° C.), while mixing using a magnetic stirrer, to make a 5% w/w of the polymer. The stiffing continued until a homogeneous clear solution was obtained (15-30 min at 85-90° C.). Then a desired amount of Starch Octenyl Succinat was added into a heated distilled water, the stiffing continued until a homogeneous clear solution was obtained. A Cocoa Butter was pre-melted at 50° C., while stiffing using a mechanical stirrer. The resulting Starch Octenyl Succinat solution and the melted CB were then homogenized into a 5% w/w of HPS solution using a homogenizer for 90 sec, the homogenizer speed was 3500 RPM (Homogenizer, HSIANG TAI MACHINERY INDUSTRY, model: HG-300 maximum speed—26000 rpm). The latter dispersion was then cooled down to 32-36° C. and kept stirred for additional 20 minutes to allow complete dissolution of the polymer.

The dispersion was finally poured into a Petrie dish (100 mm diameter) and dried at room temperature under air flow in a hood for at least 96 hours.

TABLE 2

| | HPS: CB: Starch Octenyl Succinat % | | |
|---|---|---|---|
| | (w/w) | | |
| | | | Starch |
| Films | | | Octenyl |
| Formulation | HPS | CB | Succinat |
| 100:0:0 | 100 | 0 | 0 |
| 70:0:30 | 70 | 0 | 30 |
| 60:0:40 | 60 | 0 | 40 |
| 80:10:10 | 80 | 10 | 10 |
| 70:15:15 | 70 | 15 | 15 |
| 70:10:20 | 70 | 10 | 20 |
| 70:20:10 | 70 | 20 | 10 |
| 65:25:10 | 65 | 25 | 10 |
| 60:30:10 | 60 | 30 | 10 |
| 50:40:10 | 50 | 40 | 10 |
| 50:30:20 | 50 | 30 | 20 |
| 50:25:25 | 50 | 25 | 25 |
| 50:20:30 | 50 | 20 | 30 |

Table 2 shows some embodiments of different film formulations based on different ratios of HPS: CB: Starch Octenyl Succinat.

### ii) Film Preparation: Based on HPC (Hydroxypropyl Cellulose) as the Film Forming Polymer

HPC, Cocoa Butter (CB) and Starch Octenyl Succinat were accurately weighed to obtain a certain ratio of HPC: CB:Starch Octenyl Succinat. Table 3 presents different film's formulations, possessing various ratios of HPC:CB:Starch Octenyl Succinat, prepared in the present study. First desired amount of Starch Octenyl Succinat was added into a heated distilled water, the stiffing continued until a homogeneous clear solution was obtained. A Cocoa Butter was pre-melted at 50° C., while stiffing using a mechanical stirrer. The resulting mixture was then homogenized into a 5% w/w of HPC LF dispersion which was prepared in a preheated distilled water at 75° C., as described above, using a homogenizer. The latter dispersion was then cooled down to below the LCST of the polymer and kept stirred for additional 30 minutes to allow complete dissolution of the polymer. The dispersion was finally poured into a Petrie dish (100 mm diameter) and dried at room temperature under air flow in a hood for at least 96 hours.

TABLE 3

| | HPS:CB:Starch Octenyl Succinat % | | |
|---|---|---|---|
| | (w/w) | | |
| | | | Starch |
| Films | | | Octenyl |
| Formulation | HPS | CB | Succinat |
| 100:0:0 | 100 | 0 | 0 |
| 70:15:15 | 70 | 15 | 15 |
| 70:10:20 | 70 | 10 | 20 |
| 70:20:10 | 70 | 20 | 10 |
| 70:5:25 | 70 | 5 | 25 |
| 60:20:20 | 60 | 20 | 20 |
| 50:40:10 | 50 | 40 | 10 |
| 50:30:20 | 50 | 30 | 20 |
| 50:25:25 | 50 | 25 | 25 |
| 50:20:30 | 50 | 20 | 30 |

Table 3 shows some embodiments of different film formulations based on different ratios of HPC: CB: Starch Octenyl Succinat

### iii) Water Vapor Transmission Rate (WVTR) Tests

The concept of this test is based on the measurement of the amount of water vapor transmitted through unit area of test film specimen per unit time under specified conditions. Water vapor transmission rate is expressed in grams per square meter, 24 hours [g/(m2.24 h)]. A Versaperm MkV Digital WVTR Meter of (Versaperm Ltd., Maidenhead, UK) with 10 cm² measuring area, equipped with an electrolytic detection sensor was used to measure the WVTR of different films. The method was based on ISO 15106-3:2003.

Test 1—The test was carried out at 40° C., 100% humidity for 24 hours.

Test 2—The test was carried out at Room Temperature (23-25° C., 100% humidity for 24 hours.

### iv) Preparation of Hybrid Solid Dispersion Free Films for Microencapsulation Process

HPS, Cocoa Butter (CB) and Starch Octenyl Succinat (Starch Octenyl Succinat) were accurately weighed to obtain a certain ratio of HPS: CB: Starch Octenyl Succinat. Table 4 presents different film's formulations, possessing various ratios of HPS: CB: Starch Octenyl Succinat, prepared in the present study. First a desired amount of HPS was added to a preheated distilled water (85-90° C.), while mixing using a magnetic stirrer, to make a 5% w/w of the polymer. The stiffing continued until a homogeneous clear solution was obtained (15-30 min at 85-90° C.).

Then a desired amount of Starch Octenyl Succinat was added into a heated distilled water, the stirring continued until a homogeneous clear solution was obtained. The cocoa butter was pre-melted at 50° C. while stirring using a mechanical stirrer. The resulting Starch Octenyl Succinat solution and the melted CB were then homogenized into a 5% w/w of HPS solution using a homogenizer for 90 sec, at a homogenizer speed of 3500 RPM (Homogenizer, Hsiang Tai Machinery Industry, model: HG-300 maximum speed—26000 rpm). The resulting dispersion was then cooled down to 32-36° C. and kept stirred for additional 20 minutes to allow complete dissolution of the polymer.

TABLE 4
Different film formulations based on different ratios of
HPS: CB: Starch Octenyl Succinat

| | HPS: CB: Starch Octenyl Succinat % | | |
|---|---|---|---|
| | (w/w) | | |
| | | | Starch |
| Solutions | | | Octenyl |
| Formulation | HPS | CB | Succinat |
| 55:35:10 T1 | 55 | 35 | 10 |
| 65:25:10 T1 | 65 | 25 | 10 |
| 55:35:10 T2 | 55 | 35 | 10 |
| 65:25:10 T2 | 65 | 25 | 10 |

T1—20% w/w of Bacteria from the final microcapsule. T2—10% w/w of Bacteria from the final microcapsule.

### v) Microencapsulation Preparation Process

The microencapsulation process was initiated by creating the core followed by the coating process:

### 1) Core Preparation

The core was prepared by a wet granulation method. An aqueous solution of the binder (e.g., maltodextrin) was sprayed on the powder mixture consisting of the bacteria and the filler to consolidate the particles to form agglomerates. The components used for the preparation of the microcapsules are summarized in Table 5.

TABLE 5
The composition of microcapsules according to one embodiment
of the present invention

| | | | % w/w of the |
|---|---|---|---|
| | | | final |
| Material Name | Product Name | Use | microcapsule |
| Bifidobacterialactis | Probio-Tec- | Bacteria | 10-20 |
| (Bifidobacterium) | Bifidobacterium | | |
| | Blend-100 V1 | | |
| Lactose | Pharmatose 100M | Filler | 61-67 |
| | Lactose | | |
| | Monohydrate | | |
| | (sieved) | | |
| Maltodextrin | MALDEX G190 | Binder | 12-13 |
| | PHARMA | | |
| Hydroxy propyl | EMDEN ESH 15 | Coating | 10-11 |
| starch (HPS) | | | |
| Cocoa Butter | Cocoa Butter | Coating | 4-5 |
| Starch Octenyl | Starch Octenyl | Coating | 1.8 |
| Succinate | Succinat | | |
| | (Emfix X02, K02) | | |

### 2) Coating Process

Coating took place by spraying the polymer solution directly onto the resulting core from the previous stage.

Both core preparation and the coating process were carried out at Innojet Ventilus 2.5 (Romaco-Huttlin, Esteinen-Germany)

The thickness of the layers was expressed by the % weight gain (WG) which was obtained upon the coating process in relative to the initial substrate's weight prior to the coating process according to the following equation:

$$\% WG = \frac{WG}{W 0} \times 100 = \frac{Wd - W 0}{W 0} \times 100$$

Where W_{d} and W₀ are respectively the weight of the substrate after and before coating process and WG is the weight gain.

TABLE 6
Parameters used for Bifidobacterium Granulation

| Coating Process Parameters | Data |
|---|---|
| Inlet Temperature (° C.) | 40-42 |
| Air Flow Rate (m³/hour) | 32-40 |
| Spray rate (g/min) | 3.8-4.2 |
| Product Temperature (° C.) | 38-40 |
| Upper spray Pressure (bar) | 1.85-1.9 |
| Support Air Pressure (bar) | 0.3 |

TABLE 7
Parameters used for Bifidobacterium Granules Coating

| Coating Process Parameters | Data |
|---|---|
| Inlet Temperature (0 C.) | 40-45 |
| Air Flow Rate (m3/hour) | 38-48 |
| Spray rate (g/min) | 1.8-2.2 |
| Product Temperature (0 C.) | 40 |
| Upper spray Pressure (bar) | 2.45-2.5 |

### vi) Loss On Drying (LOD) Test

During the coating process at certain times (every 4% weight gain), 2 gr of microcapsule were taken to LOD test (LOD device (MB-50-1-250, MRC)).

### vii) Stability Test

### 1) Room Conditions

Exposure of the uncoated Bifidobacterium and the microencapsulated Bifidobacterium to room conditions (variably temperature and moisture) and taking sampling to microbiology test at every week for period of 6 mounts.

### 2) Water Activity of 0.795 Aw

Exposure of the uncoated Bifidobacterium and the microencapsulated Bifidobacterium to 0.795 Aw conditions (using by desiccator with 0.795 aw solution in the bottom of the desiccator) and taking sampling to microbiology test at every 2 days for 2 weeks.

### 3) Water Activity of 0.612 Aw

Exposure of the uncoated Bifidobacterium and the microencapsulated Bifidobacterium to 0.612 Aw conditions (using by desiccator with 0.795 aw solution in the bottom of the desiccator) and taking sampling to microbiology test at every 2 days for few weeks.

### 4) Water Vapor Transmission Rate (WVTR) Tests

The concept of this test is based on the measurement of the amount of water vapor transmitted through the unit area of the test film specimen per unit time under specified conditions. The water vapor transmission rate was expressed in grams per square meter, 24 h [g/(m2, 24 h)]. A Versaperm MkV Digital WVTR Meter (Versaperm Ltd. Maidenhead, UK) with 10 cm2 measuring area, equipped with an electrolytic detection sensor was used to measure the WVTR of different films. The test was carried out at room temperature, 100% humidity for 24 h. The method was based on ISO 15106e3:2003. Briefly, the test specimen was inserted between two different chambers: a dry chamber and a controlled humidity chamber. The dry side of the specimen is swept by am flow of dry carrier gas, and water vapor permeating through the specimen from the controlled-humidity chamber is carried by the carrier gas into an electrolytic cell. This cell contains two spiral wire electrodes, which absorb quantitatively the water vapor, carried by the carrier gas, and decompose it electrolytically into hydrogen and oxygen by a D.C. voltage applied to the electrodes. The mass of the moisture which permeates through the specimen and is decomposed per unit time is calculated from the electrolytic current required.

### viii) WVTR Test Results

TABLE 8.1
WVTR Results of Different film formulations based on different
ratios of HPS: CB: Starch Octenyl Succinat

| Formula | | | Starch | | |
|---|---|---|---|---|---|
| | | Starch | Octenyl | WVTR (g/m² * day) | |
| | Cocoa | Octenyl | Succinat | Room | |
| HPS | Butter | Succinat | Type | Temperature | 40° C. |
| 60 | — | 40 | K02 | — | 407 |
| 70 | — | 30 | K02 | — | 348 |
| 80 | 20 | — | K02 | — | Disintegrated |
| 70 | 10 | 20 | K02 | — | 363 |
| 50 | 25 | 25 | K02 | — | 363 |
| 50 | 20 | 30 | K02 | — | Unable |
| 50 | 30 | 20 | K02 | — | 327 |
| 50 | 40 | 10 | K02 | — | 273 |
| 70 | 20 | 10 | X02 | — | 298 |
| 70 | 15 | 15 | X02 | — | 328 |
| 65 | 25 | 10 | X02 | — | 275 |
| 60 | 20 | 20 | X02 | 212 | — |
| 50 | 25 | 25 | X02 | — | Unable |
| 50 | 20 | 30 | X02 | — | 268 |
| 50 | 30 | 20 | X02 | — | 275 |
| 50 | 40 | 10 | X02 | — | 337 |

TABLE 8.2
WVTR Results of Different film formulations based on different
ratios of HPC-L (hydroxypropyl cellulose):
CB: Starch Octenyl Succinat Octenyl Succinat

| Formula | | | Starch | | |
|---|---|---|---|---|---|
| | | Starch | Octenyl | WVTR (g/m² * day) | |
| | Cocoa | Octenyl | Succinat | Room | |
| HPS | Butter | Succinat | Type | Temperature | 40° C. |
| 70 | 10 | 20 | K02 | — | 247 |
| 70 | 25 | 5 | K02 | — | 280 |
| 50 | 25 | 25 | K02 | 180 | — |
| 50 | 20 | 30 | K02 | 148 | — |
| 50 | 30 | 20 | K02 | 134 | 50 |
| 60 | 20 | 20 | K02 | 478 | 60 |
| 70 | 20 | 10 | X02 | 129 | 330 |
| 70 | 25 | 5 | X02 | — | 256 |
| 70 | 15 | 15 | X02 | 145 | — |
| 70 | 5 | 25 | X02 | 224 | — |
| 50 | 25 | 25 | X02 | 141 | — |
| 50 | 20 | 30 | X02 | 201 | — |
| 60 | 20 | 20 | X02 | 194 | — |

### xi) Stability Test Results

### 1) Stability During the Production Process (Microencapsulation Process Based on an Aqueous Solution of the Binder During the Granulation-Core Formation)

The microbiology test results showed that the number of bacteria, despite its high sensitivity, is not reduced during the microencapsulation process and remains the same as it was at the beginning of the process (start the process with 1.5*10¹¹ CFU/g Bifidobacterium and finish with 1.55*10¹¹ CFU/g Bifidobacterium)—at 20% weight gain (A very significant achievement).

Table 9 presents the stability results for LGG and another strain of Lactobacillus rhamnosus during the microencapsulation process (based on an aqueous solution of the binder during the core formation process).

TABLE 9
The stability (survivability) of LGG during its microencapsulation
process

| | Results (CFU/g Bacteria) | | | |
|---|---|---|---|---|
| | LGG (bacteria | | | |
| | content 10% | Lactobacillus rhamnosus | | |
| | w/w) | (bacteria content 10% w/w) | | |
| | 20% | 20% | 15% | 15% |
| | Coating: | Coating: | Coating: | Coating: |
| | (65:25:10)* | (55:35:10)* | (55:35:10)* | (55:35:10)* |
| | (10% | (10% | (10% | (15% |
| Process | Bacteria | Bacteria | Bacteria | Bacteria |
| Stage | content) | content) | content) | content) |
| Uncoated | 1.35 * 10¹¹ | 2 * 10¹¹ | 2 * 10¹¹ | — |
| Bacteria | | | | |
| Granulate | 1.45 * 10¹¹ | 2.3 * 10¹⁰ | 1.7 * 10¹⁰ | 3.1 * 10¹⁰ |
| (core) | | | | |
| Weight | | | | |
| gain | | | | |
| obtained | | | | |
| during the | | | | |
| coating | | | | |
| process | | | | |
| 2% | 1.1 * 10¹¹ | 1.5 * 10¹⁰ | 1.2 * 10¹¹ | 2.7 * 10¹⁰ |
| 4% | 1.16 * 10¹¹ | 5 * 10¹⁰ | 1 * 10¹¹ | 1.6 * 10¹⁰ |
| 6% | 5.55 * 10¹⁰ | 1.1 * 10¹¹ | 5.5 * 10¹⁰ | 1.3 * 10¹⁰ |
| 8% | 1.15 * 10¹¹ | 8.3 * 10¹⁰ | 1.7 * 10¹⁰ | 1.7 * 10¹⁰ |
| 10% | 8.2 * 10¹⁰ | 4 * 10¹⁰ | 1.6 * 10¹⁰ | 3 * 10¹⁰ |
| 12% | 3 * 10¹⁰ | 1 * 10¹¹ | 1.6 * 10¹⁰ | 1.9 * 10¹⁰ |
| 14% | 1.15 * 10¹¹ | 9.7 * 10¹⁰ | 2.5 * 10¹⁰ | 1.6 * 10¹⁰ |
| 16% | 7.9 * 10¹⁰ | 4 * 10¹⁰ | [15%] | [15%] |
| | | | 2.8 * 10¹⁰ | 1.4 * 10¹⁰ |
| 18% | 3.1 * 10¹⁰ | 5.2 * 10¹⁰ | — | — |
| 20% | 2.3 * 10¹⁰ | 4.5 * 10¹⁰ | — | — |

*Coating Formula: HPS:Cocoa Butter:Starch Octenyl Succinat X02

### 2) On-going Stability Over Time

The particles of microencapsulated bacteria as is [naked (unpackaged)] were exposed to air at room temperature (20-22° C., ≈35-45% RH) and samples were taken at different time points for the enumeration test to determine the CFU/g bacteria. The results were compared to the pure Bifidobacterium as is (non-microencapsulated or unprotected bacteria) which were kept under the same conditions. The results are summarized in Table 10.1 for up to 180 days.

Based on the results, it can clearly be noticed that Bifidobacterium as is (unprotected bacteria) losses 4-5 logs after just three weeks and remains almost unchanged over time. It is noteworthy that Bifidobacterium powder, as a result of the attachment of the bacteria to each other, creates seemingly a rigid and hard layer at the surface over time, which is manifested by a drastic change in the color (from an off white to yellowish and later to a light brown) and in touch feeling. It is believed that this new layer is responsible for the protection of the bacteria and for the diminishing in the log reduction over time. This point may be the main reason for the log's stabilization when it remains unchanged after about three weeks (10\hat{ }6).

It can also be seen that for some specific microencapsulation formulation the bacteria are totally stabilized where just only one log loss was seen after about 13 weeks. This fact indicates that the microencapsulation formulation does provide the needed protection against humidity over time.

TABLE 10.1
summarizes the results of enumeration tests done on both microencapsulated bacteria using different formulations and the pure bifidobacterial
lactis (Bifidobacterium) kept naked (unpackaged) at room temperature and ≈35-45% RH for different duration of times.

| | Results (CFU/g BB12) | | | | | | | | | |
|---|---|---|---|---|---|---|---|---|---|---|
| Sample | | 1-10 | 10-20 | 20-30 | 30-40 | 40-50 | 50-60 | 60-70 | 70-80 | 80-90 |
| Type | Fresh | Days | Days | days | days | days | days | days | days | days |
| BB12 | 1.5*10¹¹ | WBD | WBD | (27 d) | WBD | (46 d) | (55 d) | (62 d) | (76 d) | (83 d) |
| | | | | 3.55*10⁷ | | 2.3*10⁸ | 2.5*10⁷ | 6*10⁶ | 7*10⁶ | 5*10⁶ |
| HPS:Cocoa | | | | | | | | | | |
| Butter: | | | | | | | | | | |
| (50:20:30) | 1.25*10¹¹ | WBD | WBD | (27 d) | WBD | (46 d) | (55 d) | (62 d) | (76 d) | (83 d) |
| | | | | 1.15*10⁹ | | 1*10⁸ | 2*10⁷ | 2.7*10¹⁰ | 3*10⁷ | 1.8*10⁶ |
| | | | | | | | | (69 d) | | |
| | | | | | | | | 3*10⁹ | | |
| (55:35:10) | 1.45*10¹¹ | (1 d) | WBD | (20 d) | (36 d) | (43 d) | (50 d) | (64 d) | (71 d) | (85 d) |
| 10% | | 1.35*10¹¹ | | 1.0*10¹¹ | 8.65*10¹⁰ | ~5*10¹⁰ | 1.6*10¹⁰ | 2.25*10¹⁰ | 1.5*10¹⁰ | 1.0*10¹⁰ |
| coating | | | | (29 d) | | | (57 d) | | (78 d) | |
| | | | | 3.5*10¹⁰ | | | 1.1*10¹⁰ | | 1.1*10¹⁰ | |
| HPS + | 3.2*10¹⁰ | WBD | (13 d) | (21 d) | (35 d) | (42 d) | (56 d) | (63 d) | (70 d) | (84 d) |
| 10% Ta* | | | 3*10¹⁰ | 2*10¹⁰ | 5.7*10¹⁰ | 3*10⁹ | 8*10⁹ | 2*10¹⁰ | 1.5*10⁸ | 2.5*10⁸ |
| | | | | (28 d) | | (49 d) | | | (77 d) | |
| | | | | 4.5*10¹⁰ | | 6*10⁹ | | | 4*10⁸ | |
| (55:35:10) | 8*10¹⁰ | (1 d) | (15 d) | (22 d) | (36 d) | (43 d) | (50 d) | (64 d) | (71 d) | (80 d) |
| 10% | | 6.4*10¹⁰ | ~5*10¹⁰ | 2.5*10¹⁰ | 5*10¹⁰ | 8.2*10⁹ | 3*10¹⁰ | 2*10¹⁰ | 5*10¹⁰ | 4.8*10¹⁰ |
| Ta** | | (8 d) | | (29 d) | | | (57 d) | | | (87 d) |
| | | 1.2*10¹¹ | | 8.4*10¹⁰ | | | 1.8*10¹⁰ | | | 4*10¹⁰ |
| (65:25:10) | 1.1*10¹¹ | (1 d) | (16 d) | (23 d) | (32 d) | (46 d) | (53 d) | (60 d) | (76 d) | (83 d) |
| | | 8.2*10¹⁰ | 6.4*10¹⁰ | 9*10¹⁰ | 1*10¹¹ | 7.3*10¹⁰ | 8.2*10¹⁰ | 8.2*10¹⁰ | 1.4*10¹¹ | 6.5*10¹⁰ |
| | | (8 d) | | | (39 d) | | | (67 d) | | |
| | | 1.1*10¹¹ | | | 8.2*10¹⁰ | | | 5.5*10¹⁰ | | |
| (60:30:10) | 2*10¹¹ | (6 d) | (13 d) | (20 d) | (34 d) | (41 d) | (55 d) | (62 d) | (71 d) | (85 d) |
| | | 2.5*10¹¹ | 3*10¹⁰ | 1*10¹¹ | 1*10¹¹ | 6.8*10¹⁰ | 3.6*10¹⁰ | 3.5*10¹⁰ | 5*10¹⁰ | 1*10¹¹ |
| | | | | (27 d) | | (48 d) | | | (78 d) | |
| | | | | 1.4*10¹¹ | | 9.2*10¹⁰ | | | 3.8*10¹⁰ | |
| (55:35:10) | 1.75*10¹¹ | (6 d) | (13 d) | (20 d) | (34 d) | (41 d) | (55 d) | (64 d) | (71 d) | (85 d) |
| (K02) − | | 8*10¹⁰ | 6*10¹⁰ | 1.5*10¹¹ | 6.8*10¹⁰ | 1.1*10¹¹ | 3*10¹⁰ | 9.1*10¹⁰ | 9.5*10¹⁰ | 4.5*10¹⁰ |
| 10% | | | | (27 d) | | (48 d) | | | (78 d) | |
| BB12 | | | | 1.3*10¹¹ | | 5.5*10¹⁰ | | | 3.6*10¹⁰ | |
| (70:15:15) | 8.5*10¹⁰ | (8 d) | (15 d) | (22 d) | (36 d) | (43 d) | (50 d) | (66 d) | (73 d) | (80 d) |
| | | 1.2*10¹¹ | 1.5*10¹¹ | 1.36*10¹¹ | 2.5*10¹¹ | 6.8*10¹⁰ | 2.7*10¹⁰ | 4.5*10¹⁰ | 1.2*10¹¹ | 2*10¹¹ |
| | | | | (29 d) | | | (59 d) | | | (87 d) |
| | | | | 6.3*10¹⁰ | | | 1*10¹¹ | | | 2.3*10¹⁰ |
| (55:35:10) − | 2.9*10¹¹ | (3 d) | (19 d) | (26 d) | (33 d) | (40 d) | (56 d) | (63 d) | (70 d) | (83 d) |
| 20% Coating | | 3.2*10¹¹ | 1.6*10¹¹ | 3.2*10¹¹ | 3.3*10¹¹ | 1.3*10¹¹ | 3.3*10¹¹ | 2.7*10¹¹ | 3*10¹¹ | 1.6*10¹¹ |
| | | | | | | (49 d) | | | (77 d) | |
| | | | | | | 2.4*10¹¹ | | | 3*10¹⁰ | |
| (55:35:10) − | 1.1*10¹¹ | (4 d) | (13 d) | (20 d) | (36 d) | (43 d) | (50 d) | (64 d) | (71 d) | (85 d) |
| (65:25:10) − | | 1*10¹¹ | 2.8*10⁹ | 9.3*10¹⁰ | 1.35*10¹¹ | 6*10¹⁰ | 2.3*10¹¹ | 1.3*10¹¹ | 3*10¹⁰ | 3*10¹⁰ |
| 20% | | | | (27 d) | | | (57 d) | | (78 d) | |
| Coating*** | | | | 5.5*10¹⁰ | | | 1.9*10¹¹ | | 2.9*10¹⁰ | |
| (55:35:10) − | 8.2*10¹⁰ | (2 d) | (16 d) | (23 d) | (30 d) | (45 d) | (52 d) | (66 d) | (73 d) | (80 d) |
| 22% | | 6.9*10¹⁰ | 1.2*10¹¹ | 3.2*10¹¹ | 4*10¹⁰ | 1*10¹¹ | 2.7*10¹⁰ | 2.5*10¹⁰ | 2.9*10¹⁰ | 2.8*10¹⁰ |
| Coating | | (9 d) | | | (37 d) | | (59 d) | | | |
| | | 7.75*10¹⁰ | | | 4*10¹⁰ | | 2.8*10¹⁰ | | | |
| (65:25:10) − | 7.3*10¹⁰ | (2 d) | (16 d) | (23 d) | (30 d) | (44 d) | (51 d) | (65 d) | (72 d) | (86 d) |
| K02 − | | 8*10¹⁰ | 3*10¹¹ | 4*10¹⁰ | 3.3*10¹⁰ | 2.0*10¹⁰ | 3.0*10¹⁰ | 2.1*10¹⁰ | 2.3*10¹⁰ | 2.0*10¹⁰ |
| 20% | | (9 d) | | | (37 d) | | (58 d) | | | |
| Coating | | 1.4*10¹¹ | | | 2.5*10¹⁰ | | 2.8*10¹⁰ | | | |
| 25% HPS + | 5.5*10⁸ | (1 d) | (15 d) | (23 d) | (30 d) | (37 d) | (44 d) | (58 d) | (72 d) | (86 d) |
| 10% | | 4.5*10⁸ | 1.2*10⁹ | 1.3*10⁹ | 2.1*10⁹ | 1.8*10⁹ | 1.1*10¹⁰ | 2.1*10⁹ | 6.8*10⁹ | 2.6*10⁹ |
| Ta* | | (8 d) | | | | | | | (79 d) | |
| | | 1.2*10⁹ | | | | | | | 3*10⁹ | |
| (55:35:10) − | 1.45*10¹¹ | (2 d) | (11 d) | — | (31 d) | (45 d) | (52 d) | (66 d) | (73 d) | |
| HPC | | 1.45*10¹¹ | 1.1*10¹¹ | | 3.3 10¹⁰ | 2.7 10¹⁰ | 2.1 10¹⁰ | 1.8 10¹⁰ | 1.4 10¹⁰ | |
| LF − 20% | | | (17 d) | | (38 d) | | (59 d) | | | |
| Coating**** | | | 1.0*10¹¹ | | 3.1 10¹⁰ | | 2.1 10¹⁰ | | | |

| | Results (CFU/g BB12) | | | | | | | | | |
|---|---|---|---|---|---|---|---|---|---|---|
| Sample | 90-100 | 100-110 | 110-120 | 120-130 | 130-140 | 140-150 | 150-160 | 160-170 | 170-180 | 180-190 |
| Type | days | days | days | days | days | days | days | days | days | days |
| BB12 | (95 d) | (102 d) | (115 d) | (124 d) | (134 d) | | | | | |
| | 3.6*10⁶ | 3.6*10⁶ | 4.5*10⁶ | 2.6*10⁶ | 3.1*10⁶ | | | | | |
| HPS:Cocoa | | | | | | | | | | |
| Butter: | | | | | | | | | | |
| (50:20:30) | (90 d) | (104 d) | | | | | | | | |
| | 7.5*10⁶ | 6.6*10⁶ | | | | | | | | |
| | (97 d) | | | | | | | | | |
| | 3.8*10⁷ | | | | | | | | | |
| (55:35:10) | (92 d) | (108 d) | (115 d) | (122 d) | (130 d) | | | | | |
| 10% | 1.0*10¹⁰ | 7.7*10⁹ | 7*10⁹ | 6*10⁹ | 7*10⁹ | | | | | |
| coating | (99 d) | | | | | | | | | |
| | 5.5*10⁹ | | | | | | | | | |
| HPS + | (91 d) | (100 d) | (114 d) | (121 d) | (130 d) | | | | | |
| 10% Ta* | 5.5*10⁸ | 7.5*10⁹ | 2.5*10⁹ | 7.3*10⁸ | 1.2*10⁸ | | | | | |
| | | (107 d) | | | (130 d) | | | | | |
| | | 9.1*10⁸ | | | 1.3*10⁸ | | | | | |
| (55:35:10) | (94 d) | (101 d) | (115 d) | (122 d) | (136 d) | | | | | |
| 10% | 2.3*10¹⁰ | 9*10⁹ | 7.5*10⁹ | 5.2*10⁹ | 2*10⁹ | | | | | |
| Ta** | | (108 d) | | (129 d) | | | | | | |
| | | 8.5*10⁹ | | 4.8*10⁹ | | | | | | |
| (65:25:10) | (90 d) | (104 d) | (111 d) | (125 d) | (132 d) | (146 d) | (153 d) | | | |
| | 8*10¹⁰ | 2*10¹⁰ | 2.5*10¹⁰ | 1.45*10¹⁰ | 1.3*10¹⁰ | 1.2*10¹⁰ | 1.1*10¹⁰ | | | |
| | (97 d) | | (118 d) | | (139 d) | | | | | |
| | 7*10¹⁰ | | 1.5*10¹⁰ | | 1.25*10¹⁰ | | | | | |
| (60:30:10) | (92 d) | (106 d) | (113 d) | (120 d) | (134 d) | (141 d) | — | (162 d) | (176 d) | (183 d) |
| | 5.5*10¹⁰ | 1.65*10¹⁰ | 1.4*10¹⁰ | 1.2*10¹⁰ | 4*10⁹ | 3.65*10⁹ | | 1.1*10¹⁰ | 3*10⁹ | 1.9*10⁹ |
| | (99 d) | | | (127 d) | | (148 d) | | | | (190 d) |
| | 2*10¹⁰ | | | 4*10⁹ | | 1.2*10¹⁰ | | | | 2.2*10⁹ |
| (55:35:10) | (92 d) | (106 d) | (113 d) | (120 d) | (134 d) | (141 d) | (155 d) | (169 d) | (176 d) | (183 d) |
| (K02) − | 2*10¹⁰ | 1.4*10¹⁰ | 1.0*10¹⁰ | 4.5*10⁹ | 1.3*10¹⁰ | 1.6*10¹⁰ | 1.7*10¹⁰ | 4.5*10⁹ | 2.6*10⁹ | 2.5*10¹⁰ |
| 10% | (99 d) | | | (127 d) | | | | | | |
| BB12 | 2.2*10¹⁰ | | | 1.25*10¹⁰ | | | | | | |
| (70:15:15) | (94 d) | (101 d) | (115 d) | (122 d) | (136 d) | — | (150 d) | (164 d) | (171 d) | (185 d) |
| | 1.8*10¹⁰ | 1.7*10¹⁰ | 1.4*10¹⁰ | 6*10⁹ | 2.25*10¹⁰ | | 2.0*10¹⁰ | 9.1*10⁹ | 3*10⁹ | 6*10⁹ |
| | | (108 d) | | (129 d) | | | | | (178 d) | |
| | | 1.3*10¹⁰ | | 1.6*10¹⁰ | | | | | 3.5*10⁹ | |
| (55:35:10) − 20% | (90 d) | (104 d) | (111 d) | (125 d) | (139 d) | (153 d) | (160 d) | (174 d) | (181 d) | (195 d) |
| Coating | 1.15*10¹¹ | 1.7*10¹¹ | 2*10¹¹ | 1.6*10¹¹ | 1.3*10¹¹ | 1.3*10¹¹ | 1.2*10¹¹ | 1.4*10¹¹ | 1.1*10¹¹ | 3.2*10¹⁰ |
| | (97 d) | | (118 d) | | | | (167 d) | | (188 d) | |
| | 1.5*10¹¹ | | 1.5*10¹¹ | | | | 1.4*10¹¹ | | 9*10¹⁰ | |
| (55:35:10) − | (92 d) | (106 d) | (113 d) | (127 d) | (141 d) | (155 d) | (162 d) | (176 d) | (183 d) | |
| (65:25:10) − | 2.8*10¹⁰ | 2.4*10¹⁰ | 2.6*10¹⁰ | 2.6*10¹⁰ | 2.2*10¹⁰ | 1.5*10¹⁰ | 1.5*10¹⁰ | 1.2*10¹⁰ | 9*10⁹ | |
| 20% | (99 d) | | | | (148 d) | | (169 d) | | | |
| Coating*** | 2.9*10¹⁰ | | | | 2.7*10¹⁰ | | 1.5*10¹⁰ | | | |
| (55:35:10) − | (94 d) | (108 d) | (115 d) | (122 d) | (136 d) | (143 d) | (150 d) | | | |
| 22% | 2.5*10¹⁰ | 2.45*10¹⁰ | 2.6*10¹⁰ | 1.5*10¹⁰ | 1.1*10¹⁰ | 1.4*10¹⁰ | 1.3*10¹⁰ | | | |
| Coating | | | | (129 d) | | | | | | |
| | | | | 1.9*10¹⁰ | | | | | | |
| (65:25:10) − | — | (100 d) | (114 d) | (128 d) | (135 d) | (143 d) | | | | |
| K02 − | | 2.45*10¹⁰ | 2.3*10¹⁰ | 1.6*10¹⁰ | 1.8*10¹⁰ | 1.4*10¹⁰ | | | | |
| 20% | | | | | | | | | | |
| Coating | | | | | | | | | | |
| 25% HPS + | (93 d) | (100 d) | (113 d) | | | | | | | |
| 10% | 3*10⁹ | 2.3*10⁹ | 1.8*10⁹ | | | | | | | |
| Ta* | | (107 d) | | | | | | | | |
| | | 3.8*10⁹ | | | | | | | | |
| (55:35:10) − | | | | | | | | | | |
| HPC | | | | | | | | | | |
| LF − 20% | | | | | | | | | | |
| Coating**** | | | | | | | | | | |

*The Coating Solution Formula: only HPS and tannic acid (Ta)
**The coating contained additionally Ta
***Double-layer coating, 55:35:10 as the inner layer coating and 65:25:10 as the outer coating layer
****HPC LF was used instead of HPS

TABLE 10.2
The effect of Starch Octenyl Succinat type on stability of the microencapsulated bifidobacterial lactis (Bifidobacterium) kept naked
(unpackaged) at room temperature and ≈35-45% RH for different duration of times compared to the neat bacteria (non-microencapsulated).

| | Results (CFU/g BB12) | | | | | | | | | |
|---|---|---|---|---|---|---|---|---|---|---|
| Sample/ | | 1-10 | 10-20 | 20-30 | 30-40 | 40-50 | 50-60 | 60-70 | 70-80 | 80-90 |
| Formula | Fresh | Days | Days | days | days | days | days | days | days | days |
| BB12 | 1.5*10¹¹ | — | — | (27 d) | — | (46 d) | (55 d) | (62 d) | (76 d) | (83 d) |
| | | | | 3.55*10⁷ | | 2.3*10⁸ | 2.5*10⁷ | 6*10⁶ | 7*10⁶ | 5*10⁶ |
| (55:35:10)* − | 1.45*10¹¹ | (1 d) | — | (20 d) | (36 d) | (43 d) | (50 d) | (64 d) | (71 d) | (85 d) |
| X02 | | 1.35*10¹¹ | | 1.0*10¹¹ | 8.65*10¹⁰ | ~5*10¹⁰ | 1.6*10¹⁰ | 2.25*10¹⁰ | 1.5*10¹⁰ | 1.0*10¹⁰ |
| | | | | (29 d) | | | (57 d) | | (78 d) | |
| | | | | 3.5*10¹⁰ | | | 1.1*10¹⁰ | | 1.1*10¹⁰ | |
| (55:35:10) − | 1.75*10¹¹ | (6 d) | (13 d) | (20 d) | (34 d) | (41 d) | (55 d) | (64 d) | (71 d) | (85 d) |
| K02 | | 8*10¹⁰ | 6*10¹⁰ | 1.5*10¹¹ | 6.8*10¹⁰ | 1.1*10¹¹ | 3*10¹⁰ | 9.1*10¹⁰ | 9.5*10¹⁰ | 4.5*10¹⁰ |
| | | | | | | | | | (78 d) | |
| | | | | | | | | | 3.6*10¹⁰ | |

| | Results (CFU/g BB12) | | | | | | |
|---|---|---|---|---|---|---|---|
| Sample/ | 90-100 | 100-110 | 110-120 | 120-130 | 130-140 | 140-150 | 150-160 |
| Formula | days | days | days | days | days | days | days |
| BB12 | (95 d) | (102 d) | (115 d) | (124 d) | (134 d) | WBD | WBD |
| | 3.6*10⁶ | 3.6*10⁶ | 4.5*10⁶ | 2.6*10⁶ | 3.1*10⁶ | | |
| (55:35:10)* − | (92 d) | (108 d) | (115 d) | (122 d) | (130 d) | WBD | WBD |
| X02 | 1.0*10¹⁰ | 7.7*10⁹ | 7*10⁹ | 6*10⁹ | 7*10⁹ | | |
| | (99 d) | | | | | | |
| | 5.5*10⁹ | | | | | | |
| (55:35:10) − | (92 d) | (106 d) | (113 d) | (120 d) | (134 d) | (141 d) | (155 d) |
| K02 | 2*10¹⁰ | 1.4*10¹⁰ | 1.0*10¹⁰ | 4.5*10⁹ | 1.3*10¹⁰ | 1.6*10¹⁰ | 1.7*10¹⁰ |
| | (99 d) | | | (127 d) | | | |
| | 2.2*10¹⁰ | | | 1.25*10¹⁰ | | | |

*The Coating Solution Formula: HPS:Cocoa Butter:SOS (Starch Octenyl Succinat)

TABLE 10.2
The results of enumeration tests done on both microencapsulated bacteria and pure LGG kept
naked (unpackaged) at room temperature and ≈35-45% RH for different duration of times.

| Sample | Table 10.2 - Results (CFU/g Bacteria) | | | | | | | |
|---|---|---|---|---|---|---|---|---|
| Type | Fresh | 10 | 20 | 30 | 40 | 50 | 60 | 70 |
| Uncoated | 1.35*10¹¹ | 1.2*10¹¹ | 1.5*10¹⁰ | 5.5*10⁹ | 1*10⁹ | 1.1*10⁹ | 9.5*10⁸ | 3.2*10⁸ |
| LGG | | | | | | | | |
| (65:25:10) − | 2.3*10¹⁰ | 1.2*10¹⁰ | 3.2*10¹⁰ | 4*10¹⁰ | 2.2*10¹⁰ | 2.8*10¹⁰ | 2.2*10¹⁰ | 2.1*10¹⁰ |
| 20% | | | | | | | | |

| Sample | Table 10.2 - Results (CFU/g Bacteria) | | | | | |
|---|---|---|---|---|---|---|
| Type | 80 | 90 | 100 | 110 | 120 | 130 |
| Uncoated | 1.2*10⁶ | 1.2*10⁶ | 4.5*10⁷ | 2.4*10⁴ | 1.3*10⁶ | 0 |
| LGG | | | | | | |
| (65:25:10) − | 1.4*10¹⁰ | 8*10⁸ | 8.6*10⁸ | 1.3*10⁹ | 3*10⁹ | 1.3*10⁹ |
| 20% | | | | | | |

TABLE 10.3
Stability of BB12 and Microencapsulated BB12 during time at AW of 0.612.

| Sample | Results (CFU/g BB12) | | | | | |
|---|---|---|---|---|---|---|
| Type | Fresh | 2 day | 4 day | 7 day | 9 day | 11 day |
| BB12 | 1.5*10¹¹ | 3.2*10¹⁰ | 3.6*10¹⁰ | 2*10⁸ | 4*10⁷ | 4.7*10⁵ |
| (55:35:10) | 1.45*10¹¹ | 7.3*10¹⁰ | 5.6*10¹⁰ | 1.5*10¹⁰ | 1*10¹⁰ | 4.8*10⁸ |
| (65:25:10) | 1.1*10¹¹ | 1.1*10¹¹ | 8.6*10¹⁰ | 1.4*10¹⁰ | 4.8*10⁹ | 9*10⁸ |
| (55:35:10) − | 3.2*10¹¹ | 1.1*10¹¹ | 2.2*10¹¹ | 1.9*10¹¹ | 5*10¹⁰ | 2*10¹⁰ |
| 20% | | | | | | |
| Coating | | | | | | |
| (65:25:10) − | 7.3*10¹⁰ | 9*10¹⁰ | 5*10¹⁰ | 2.7*10¹⁰ | 1.1*10¹⁰ | 2.5*10⁹ |
| K02 − | | | | | | |
| 20% | | | | | | |
| Coating | | | | | | |

| Sample | Results (CFU/g BB12) | | | | |
|---|---|---|---|---|---|
| Type | 14 day | 16 day | 18 day | 21 day | 25 day |
| BB12 | 1.3*10⁴ | 1.55*10⁵ | 5.4*10² | 2*10⁵ | — |
| (55:35:10) | 5.5*10⁸ | 7*10⁶ | 7.7*10⁶ | 1*10⁵ | — |
| (65:25:10) | 1*10⁸ | 1.1*10⁷ | 1.6*10⁷ | 5.5*10⁵ | — |
| (55:35:10) − | 2*10⁹ | 2*10⁸ | 2*10⁷ | 1.2*10⁷ | 2.2*10⁷ |
| 20% | | | | | |
| Coating | | | | | |
| (65:25:10) − | 2.5*10⁸ | 2.4*10⁷ | 7*10⁶ | 1.4*10⁵ | 9*10⁶ |
| K02 − | | | | | |
| 20% | | | | | |
| Coating | | | | | |

TABLE 10.4
Stability of BB12 and Microencapsulated BB12 during time at AW of 0.795

| Sample | Results (CFU/g BB12) | | | | | | | | | | |
|---|---|---|---|---|---|---|---|---|---|---|---|
| Type | Fresh | 1 day | 2 day | 3 day | 4 day | 7 day | 8 day | 9 day | 10 day | 11 day | 14 day |
| BB12 | 1.5*10¹¹ | 1.3*10¹¹ | 1.1*10¹¹ | 1*10¹¹ | 9.5*10⁹ | 7.3*10⁷ | 3*10⁶ | 8.2*10⁷ | 3*10⁴ | 6.8*10⁴ | 6.8*10³ |
| | 1.5*10¹¹ | 5.6*10¹⁰ | 2.7*10⁹ | 6*10⁴ | 2.5*10⁵ | 2*10⁴ | — | 5.5*10⁴ | | | |
| (50:20:30) | 1.25*10¹¹ | 1.6*10¹¹ | 9.55*10¹⁰ | 8.2*10¹⁰ | 4.5*10⁹ | 1.4*10⁸ | 1.2*10⁷ | 5.9*10⁵ | 2.5*10⁵ | 5.4*10³ | 4.1*10⁴ |
| (55:35:10) | 1.45*10¹¹ | 2.9*10¹¹ | 1.3*10¹¹ | 8.4*10¹⁰ | 4.1*10¹⁰ | 4*10⁹ | 1*10⁹ | 1.4*10⁸ | 3.4*10⁶ | 1*10⁵ | 1.3*10³ |
| HPS + 10% | 3.2*10¹⁰ | 3.2*10¹⁰ | 1.1*10¹⁰ | 3.4*10¹⁰ | 4.3*10⁸ | 4*10⁷ | 2.3*10⁶ | 1.6*10⁴ | 9.1*10³ | 0 | 0 |
| Ta | | | | | | | | | | | |
| (55:35:10) | 8*10¹⁰ | 1.2*10¹¹ | 7*10¹⁰ | 6.4*10¹⁰ | 5.9*10⁹ | 7.75*10⁸ | 1.2*10⁶ | 1.2*10⁶ | 5*10⁴ | 0 | 0 |
| 10% Ta | | | | | | | | | | | |
| (65:25:10) | 1.1*10¹¹ | 1.6*10¹¹ | 1.2*10¹¹ | 9.3*10¹⁰ | 1.4*10¹⁰ | 8.2*10⁸ | 3*10⁸ | 3.6*10⁷ | 1*10⁶ | 7.7*10⁴ | 3.4*10⁴ |
| (60:30:10) | 2*10¹¹ | 1.15*10¹¹ | 1.2*10¹¹ | 8.2*10¹⁰ | 2*10¹⁰ | 2.5*10¹¹ | 2.8*10⁸ | 2.1*10⁸ | 1.4*10⁷ | 2.8*10⁵ | 9*10³ |
| (70:15:15) | 8.5*10¹⁰ | 1.6*10¹¹ | 1.1*10¹¹ | 3*10¹⁰ | 1.45*10¹⁰ | 2.2*10⁹ | 2*10⁸ | 2*10⁸ | 1.4*10⁷ | 1*10⁶ | 1.8*10⁵ |
| (55:35:10) − | 3.2*10¹¹ | 2.25*10¹¹ | 1.4*10¹¹ | 2.9*10¹¹ | 2.45*10¹¹ | 1.1*10¹⁰ | 2.3*10⁹ | 1.6*10⁹ | 4*10⁷ | 1.8*10⁶ | 6.3*10² |
| 20% | | | | | | | | | | | |
| Coating | | | | | | | | | | | |
| (55:35:10) − | 1.1*10¹¹ | 2.3*10¹¹ | 9.1*10¹⁰ | 1.5*10¹¹ | 2.3*10¹⁰ | 2.8*10⁹ | 1.3*10⁹ | 1.9*10⁶ | 1.65*10⁸ | 2.7*10⁸ | 0 |
| (65:25:10) − | | | | | | | | | | | |
| 20% | | | | | | | | | | | |
| Coating | | | | | | | | | | | |
| (55:35:10) | 1.75*10¹¹ | 5*10¹⁰ | 1.5*10¹¹ | 3*10⁹ | 1.3*10⁹ | 6.4*10⁷ | 6.4*10⁶ | 1.4*10⁷ | 6.4*10⁴ | 5*10⁴ | 2*10⁶ |
| (K02) | | | | | | | | | | | |
| (55:35:10) − | 8.2*10¹⁰ | 1.1*10¹¹ | 2.3*10¹⁰ | 5*10⁹ | 5*10⁹ | 2.3*10⁸ | 1.1*10⁸ | 1.1*10⁷ | 2*10⁶ | 1.2*10⁴ | 1*10⁵ |
| 22% | | | | | | | | | | | |
| Coating | | | | | | | | | | | |

[symbol]

### 4) Water Content Measurement Over Time

The water content of the microencapsulated bacteria with a certain formulation compared to the pure bacteria, kept at the same conditions where the on-going stability test is being performed, was also measured over time using a Loss-On-Dry (LOD) method. Note that any increase in the water content can indicate water absorption taking place by the powder and thus a poor barrier capability. Contrastively, no change in LOD value may signify a good protection against humidity.

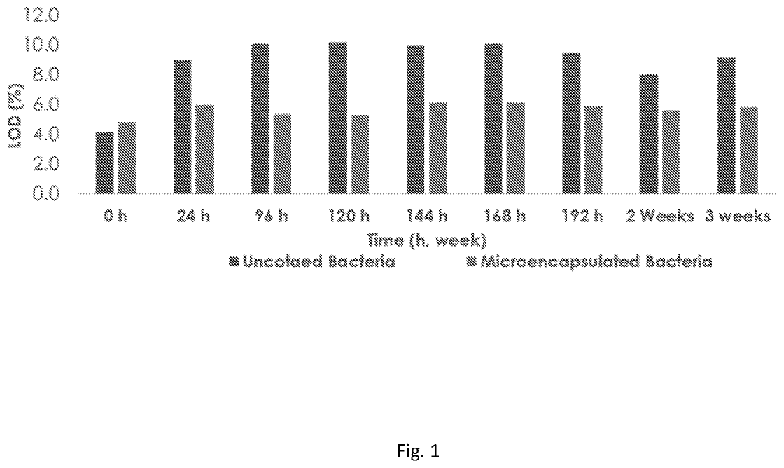

The results of water content are summarized in Table 11 and graphically illustrated in FIG. 1 for BB-12.

TABLE 11.1
the water content measured (humidity uptake) in microencapsulated
Bifidobacterium kept at room temperature, ≈35-45%
RH over time compared to Bifidobacterium as is.

| | Table 11.1 - LOD (%) | | | | | | | | |
|---|---|---|---|---|---|---|---|---|---|
| Sample | 0 | 24 | 96 | 120 | 144 | 168 | 192 | Week | Week |
| Description | h | h | h | h | h | h | h | 2 | 3 |
| Bifidobacterium | 4.2 | 9.0 | 10.1 | 10.2 | 10.0 | 10.1 | 9.5 | 8.0 | 9.2 |
| Microencapsulated | 4.8 | 5.9 | 5.4 | 5.3 | 6.1 | 6.1 | 5.9 | 5.6 | 5.8 |
| Bifidobacterium | | | | | | | | | |
| Batch# 30082017 | | | | | | | | | |

Reference is now made to FIG. 1 which depicts the water content of the microencapsulated bacteria with a certain formulation compared to the pure bacteria, kept at the same conditions where the on-going stability test is being performed, was also measured over time using a Loss-On-Dry (LOD) method.

As shown in FIG. 1 the microencapsulation formulation provides a proper barrier against moisture absorption and consequently no significant increase in the water content of the microencapsulated Bifidobacterium could be observed over time. In contrast, unprotected bacteria after 24 hour-exposure to air absorbed about 114% moisture which remains almost constant over time.

The same experiment was performed on both unprotected (LGG as is) and microencapsulated LGG and the results are demonstrated in Table 11.2.

TABLE 11.2
The water content (humidity uptake) measured in microencapsulated LGG
kept at room temperature, ≈35-45% RH over time compared to LGG as is.

| | LOD (%) | | | | | | | |
|---|---|---|---|---|---|---|---|---|
| Sample | Time | Week | Week | Week | Week | Week | Week | Week |
| Type | 0 | 1 | 2 | 3 | 4 | 5 | 6 | 7 |
| Uncoated | 4.8 | 12.8 | 13 | 12.6 | 11.5 | 11.2 | 11 | 12.6 |
| L. rhamnosus | | | | | | | | |
| Microencapsulated | 4.6 | 4.5 | 4.2 | 5 | 3.9 | 5.2 | 4.2 | 4.4 |
| L. rhamnosus | | | | | | | | |

While this invention has been described in terms of some specific examples, many modifications and variations are possible. It is therefore understood that within the scope of the appended claims, the invention may be realized otherwise than as specifically described.

### Example 2—Core and Coating Composition Embodiments—(melting-Based Core Formation)

The core was prepared by a melt granulation method. A melted liquid of the binder (e.g., Cocoa Butter) was sprayed on the powder mixture consisting of the bacteria and the filler to consolidate the particles to form agglomerates. The components used for the preparation of the microcapsules are summarized in Table 12.

TABLE 12
The composition of microcapsules according to one embodiment of the present invention

| | | | | % w/w |
|---|---|---|---|---|
| | | | | content in the |
| | | | | final |
| Material Name | Product Name | Supplier | Use | microcapsule |
| Lactobacillus | Lactobacillus | | Bacteria | 10-60 |
| Rhamnosus | rhamnosus | | | |
| Lactose | Pharmatose 100M | DFE Pharma | Filler | 25-67 |
| | Lactose | | | |
| | Monohydrate | | | |
| | (sieved) | | | |
| Maltodextrin | MALDEX G190 | TEROSE SYRAL | Filer | 0-5 |
| | PHARMA | | | |
| HPS | EMDEN ESH 15 | EMSLAND | Coating | 10-11 |
| | | GROUP | | |
| Cocoa Butter | Cocoa Butter | Lubeca Germany | Coating/Melt | 4-15 |
| | | | Binder | |
| Starch Octenyl | Starch Octenyl | EMSLAND | Coating | 1.8 |
| Succinate | Succinat X02, K02 | GROUP | | |

TABLE 13
Parameters used for Lactobacillus rhamnosus melt granulation

| Coating Process Parameters | Data |
|---|---|
| Inlet Temperature (° C.) | Room Temperature |
| Air Flow Rate (m³/hour) | 25-40 |
| Liquid Pump (RPM) | 35-70 |
| Product Temperature (° C.) | 30-32 |
| Upper spray Pressure (bar) | 0.5-0.8 |
| Support Air Pressure (bar) | — |

TABLE 14
Parameters used for Lactobacillus rhamnosus granules coating

| Coating Process Parameters | Data |
|---|---|
| Inlet Temperature (° C.) | 34-40 |
| Air Flow Rate (m3/hour) | 38-60 |
| Spray rate (g/min) | 1.8-2.2 |
| Product Temperature (0 C.) | 34-39 |
| Upper spray Pressure (bar) | 2.45-2.5 |

### Stability During the Production Process

Samples were taken for enumeration test to determine the stability of the bacteria (Lactobacillus rhamnosus) during the microencapsulation process. The results are summarized in Table 15.

TABLE 15
The stability (survivability) of Lactobacillus rhamnosus during its microencapsulation process
using a melt granulation process.

| | Results (CFU/ g Bacteria) | | | |
|---|---|---|---|---|
| | 10% Coating: | 16% Coating: | 10% Coating: | 10% Coating: |
| | (55:35:10)* | (55:35:10)* | (55:35:10)* | (55:35:10)** |
| | [Bacteria | [Bacteria | [Bacteria | [Bacteria |
| Process | content | content | content | content |
| Stage | 10% w/w] | 35% w/w] | 50% w/w] | 50% w/w] |
| Uncoated | 2.4 * 10¹¹ | 2.4 * 10¹¹ | 2.4 * 10¹¹ | 2.4 * 10¹¹ |
| Bacteria | | | | |
| Granulate | 1.6 * 10¹¹ | 3.0 * 10¹¹ | 3.0 * 10¹¹ | 1.8 * 10¹¹ |
| Weight gain | | | | |
| during the | | | | |
| coating | | | | |
| process | | | | |
| 2% | 8.5 * 10¹⁰ | 1.1 * 10¹¹ | 3.0 * 10¹¹ | 1.1 * 10¹¹ |
| 4% | 6.9 * 10¹⁰ | 8.4 * 10¹⁰ | 3.3 * 10¹¹ | 6.8 * 10¹⁰ |
| 6% | 7.0 * 10¹⁰ | 8.5 * 10¹⁰ | 2.5 * 10¹¹ | 1.2 * 10¹¹ |
| 8% | 8.6 * 10¹⁰ | 9.1 * 10¹⁰ | 2.4 * 10¹¹ | 1.2 * 10¹¹ |
| 10% | 5.1 * 10¹⁰ | 8.3 * 10¹⁰ | 1.8 * 10¹¹ | 1.8 * 10¹¹ |
| 12% | — | 7.2 * 10¹⁰ | — | |
| 14% | — | 5.5 * 10¹⁰ | — | |
| 16% | — | 5.0 * 10¹⁰ | — | |
| 18% | — | — | — | |
| 20% | — | — | — | |

*Coating Formula: HPS : Cocoa Butter: Starch Octenyl Succinat X02
** Coating Formula: HPS : Stearic acid: Starch Octenyl Succinat X02

While this invention has been described in terms of some specific examples, many modifications and variations are possible. It is therefore understood that within the scope of the appended claims, the invention may be realized otherwise than as specifically described.

What is claimed is:

1. A probiotic microcapsule comprising:
a core comprising probiotic microorganisms; and,
a coating layer comprising a hybrid solid dispersion comprising an edible fatty molecule evenly dispersed within a water-soluble film forming polymer and an edible mediator, wherein said edible mediator is starch octenyl succinate.

2. The probiotic microcapsule of claim 1, wherein said water-soluble film forming polymer is hydroxypropyl starch.

3. The probiotic microcapsule of claim 2, wherein said probiotic microorganisms comprise Bifidobacterium.

4. The probiotic microcapsule of claim 3, wherein said edible fatty molecule is selected from the group including lauric acid, myristic acid, palmitic acid, palmitate, palmitoleate, hydroxypalmitate, arachidic acid, oleic acid, stearic acid, sodium stearat, calcium stearate, magnesium stearate, hydroxyoctacosanyl hydroxystearate, oleate esters of long-chain, esters of fatty acids, fatty alcohols, esterified fatty diols, hydroxylated fatty acid, hydrogenated fatty acid (saturated or partially saturated fatty acids), partially hydrogenated soybean, partially hydrogenated cottonseed oil, aliphatic alcohols, phospholipids, lecithin, phosphathydil cholin, triesters of fatty acids, coconut oil, hydrogenated coconut oil, cacao butter; palm oil; fatty acid eutectics; mono and diglycerides, poloxamers, block-co-polymers of polyethylene glycol and polyesters or a combination thereof.

5. The probiotic microcapsule of claim 4, wherein said edible fatty molecule is cocoa butter.

6. The probiotic microcapsule of claim 4, wherein said edible fatty molecule is stearic acid.

7. The probiotic microcapsule of claim 1, wherein said hybrid solid dispersion is a single hybrid solid dispersion.

8. A method for preparing a microcapsule comprising:
preparing a microcapsule core comprising Bifidobacterium;
preparing at least one coating comprising low water vapor transmission rate (WVTR) values comprising Hydroxypropyl starch (HPS) Cocoa Butter (CB) and Starch Octenyl Succinat; and
coating said core with said at least one coating.

9. The method of claim 8, wherein preparing at least one coating comprising
preparing a 5% w/w solution of Hydroxypropyl starch (HPS) by adding HPS to heated distilled water (85-90° C.);
preparing a solution of Starch Octenyl Succinat (SOS) by adding SOS to heated distilled water and stirring continually until a homogeneous clear solution is obtained;
pre-melting Cocoa Butter (CB) while stiffing using a mechanical stirrer to provide melted CB; and
homogenizing said SOS solution and said melted CB into said 5% w/w solution of HPS using a homogenizer.

10. The method of claim 9, wherein pre-melting said Cocoa Butter (CB) is performed at 50° C.
